(19) United States
(12) Patent Application Publication
(10) Pub. No.: US 2022/0073848 A1
(43) Pub. Date: Mar. 10, 2022

(54) TUBULAR VITRIGEL AND USE THEREOF

(71) Applicant: National Agriculture and Food Research Organization, Tsukuba-shi, Ibaraki (JP)

(72) Inventor: Toshiaki TAKEZAWA, Tsukuba-shi (JP)

(21) Appl. No.: 17/417,252

(22) PCT Filed: Nov. 25, 2019
(86) PCT No.: PCT/JP2019/045992

§ 371 Date: Jun. 22, 2021

(30) Foreign Application Priority Data
Dec. 26, 2018 (JP) 2018-243681

(51) Int. Cl. C12M 3/00; C12N 5/00; C12M 1/12; A61L 27/52; A61L 27/54; A61L 27/24; B32B 3/26; B32B 9/02; B32B 9/04

(52) U.S. Cl. CPC C12M 21/08; C12N 5/0012; C12M 23/06; A61L 27/52; B32B 2535/00; A61L 27/24; B32B 3/266; B32B 9/02; B32B 9/04; A61L 27/54

(57) ABSTRACT

There is provided a tubular vitrigel composed of a laminate in which a plurality of plate-like vitrigels each having a through-hole are laminated in a thickness direction so that the through-holes are continuous.

### TECHNICAL FIELD

The present invention relates to a tubular vitrigel (registered trademark) and a use thereof. More specifically, the present invention relates to a tubular vitrigel (registered trademark), a device for producing a tubular vitrigel (registered trademark), a method of producing a tubular vitrigel (registered trademark), an artificial tubular tissue, a cell encapsulation device and a cell encapsulation component. Here, in this specification, when the term “vitrigel” is used, the term “(registered trademark)” may be omitted.

Priority is claimed on Japanese Patent Application No. 2018-243681, filed Dec. 26, 2018, the content of which is incorporated herein by reference.

### BACKGROUND ART

A technology in which a collagen sol is injected into a container, an optimal salt concentration, pH, and temperature are applied to form a gel, and the gel is then sufficiently dried to vitrify and also rehydrate, and thus the collagen gel is converted into a thin film with excellent strength and transparency with favorable reproducibility has been developed. In the case of a hydrogel, a gel having a component other than collagen is also vitrified and then rehydrated, and thus the gel can be converted into a gel having new stable physical properties. The gel having new physical properties produced through a vitrification process is called vitrigel (registered trademark) (for example, refer to Patent Literature 1).

Patent Literature 1 describes a method of producing a vitrigel having a desired shape in which a hydrogel having a desired shape other than a thin film shape is dehydrated, dried and vitrified at an angle formed in the long axis direction of the shape of the hydrogel with respect to a horizontal direction.

### CITATION LIST

### Patent Literature

### [Patent Literature 1]

Japanese Patent No. 4677559

### SUMMARY OF INVENTION

### Technical Problem

However, in the production method described in Patent Literature 1, a hydrogel formed from the sol from which water is removed is vitrified and then rehydrated to produce a vitrigel. Therefore, in the case of a tubular vitrigel, if the thickness of the tube is thicker, a larger amount of water needs to be removed from the hydrogel, which makes production difficult. In addition, the production of a tubular vitrigel having an inner diameter of less than 4 mm has a drawback that it is very difficult to remove water from the lumen. In addition, in the production method described in Patent Literature 1, since it is necessary to set an angle formed in the long axis direction of the shape of the hydrogel with respect to a horizontal direction, it is not possible to produce a tubular vitrigel using a hydrogel such as atelocollagen, which is inferior in strength to native collagen. Here, an object of the present invention is to provide a new technology for producing a tubular vitrigel using a plate-like vitrigel produced in advance.

### Solution to Problem

The present invention includes the following aspects.

[1] A tubular vitrigel composed of a laminate in which a plurality of plate-like vitrigels each having a through-hole are laminated in a thickness direction so that the through-holes are continuous.
[2] The tubular vitrigel according to [1], wherein irregularities of the outer surface of the tube are larger than irregularities of the inner surface of the tube.
[3] The tubular vitrigel according to [1] or [2], wherein the inner diameter of the tube is 5 μm or more.
[4] A tubular vitrigel obtained by additionally winding a sheet-like vitrigel around an outer circumference of the tubular vitrigel according to any one of [1] to [3].
[5] The tubular vitrigel according to any one of [1] to [4], containing native collagen or atelocollagen.
[6] The tubular vitrigel according to any one of [1] to [5], containing a functional material in at least a part.
[7] The tubular vitrigel according to [6], wherein the functional material includes a support, a magnetic substance, a fluorescent material, a contrast agent, an immunosuppressive agent, an anti-inflammatory agent, and a differentiation-inducing factor.
[8] A device for producing the tubular vitrigel according to any one of [1] to [7], including a base and a columnar structure that is able to be fixed to the base.
[9] The production device according to [8], wherein the outer diameter of the columnar structure is 5 μm or more.
[10] The production device according to [8] or [9], wherein a pedestal having an outer diameter larger than that of the columnar structure is additionally provided at a foundation of the columnar structure.
[11] A method of producing a tubular vitrigel, including: a process (a) in which, on the columnar structure of the production device according to any one of [8] to [10], (i) after a sol is applied to surfaces of a plurality of plate-like vitrigels each having a through-hole, the vitrigels are laminated through the through-holes, or (ii) after a plurality of plate-like vitrigels each having a through-hole are laminated through the through-holes, a sol is applied to surfaces, to obtain a plate-like vitrigel laminate having a surface coated with the sol; a process (b) in which the sol is gelled to obtain a tubular hydrogel in which plate-like vitrigels constituting the laminate are connected; a process (c) in which the tubular hydrogel is dried to obtain the tubular hydrogel as a dried component; and a process (d) in which the tubular hydrogel dried component is rehydrated and removed from the columnar structure to obtain a tubular vitrigel.
[12] The production method according to [11], further including a process in which ultraviolet rays are irradiated to the tubular hydrogel dried component after the process (c) and before the process (d).
[13] The production method according to [11] or [12], further including a process in which a sheet-like hydrogel having a surface coated with a sol is wound around the laminate after the process (a) and before the process (b), or a process in which a sheet-like hydrogel having a surface coated with a sol is wound around the tubular hydrogel, and the sol is gelled to obtain a tubular hydrogel after the process (b) and before the process (c), or a process in which a sheet-like hydrogel having a surface coated with a sol is wound around the tubular hydrogel dried component, the sol is gelled to obtain a tubular hydrogel, and the tubular hydrogel is dried to obtain the tubular hydrogel as a dried component after the process (c) and before the process (d).
[14] An artificial tubular tissue composed of the tubular vitrigel according to any one of [1] to [7].
[15] A cell encapsulation device composed of the tubular vitrigel according to any one of [1] to [7].
[16] A cell encapsulation component in which a liquid containing cells is retained in the central part of the cell encapsulation device according to [15] in a long axis direction, and air is present at both end parts in the long axis direction.

### Advantageous Effects of Invention

According to the present invention, it is possible to provide a new technology for producing a tubular vitrigel.

### BRIEF DESCRIPTION OF DRAWINGS

FIGS. 1(a) to 1(c) are schematic views illustrating a structure of an example of a tubular vitrigel.

FIGS. 2(a) to 2(c) are schematic views illustrating a structure of an example of a tubular vitrigel.

FIGS. 3(a) and 3(b) are perspective views showing an example of each device for producing a tubular vitrigel. FIG. 3(c) is a cross-sectional view taken along the line c-c′ in FIG. 3(b). FIG. 3(d) is a cross-sectional view showing a state in which a plurality of plate-like hydrogels are penetrated and laminated on a columnar structure of a production device.

FIG. 4 is a schematic cross-sectional view showing a state in which a liquid is retained inside the tubular vitrigel.

FIG. 5 is a schematic view showing a structure of the production device used in Production Example 1.

FIGS. 6(a) to 6(h) are images showing procedures of Experimental Example 1.

FIGS. 7(a) to 7(n) are images showing procedures of Experimental Example 2.

FIGS. 8(a) to 8(p) are images of tubular vitrigels of Experimental Examples 1 to 4.

FIGS. 9(a) to 9(e) are images showing procedures of Experimental Example 5.

FIGS. 10(a) to 10(c) are images showing procedures of Experimental Example 6.

FIGS. 11(a) to 11(c) are images showing procedures of Experimental Example 7.

FIGS. 12(a) to 12(c) are images showing procedures of Experimental Example 8.

FIG. 13(a) is an image showing a state in which a cell encapsulation component floats in a culture medium in Experimental Example 9. FIG. 13(b) is an enlarged image of FIG. 13(a). FIG. 13(c) is an image showing a state 5 minutes after a cell encapsulation component floats in a culture medium in Experimental Example 9. FIG. 13(d) is an enlarged image of FIG. 13(c).

FIGS. 14(a) to 14(d) are microscopic images showing the results obtained by observing a cell encapsulation component 15 minutes after culturing under a phase contrast microscope in Experimental Example 9.

FIG. 15 is a microscopic image showing the result obtained by observing a cell encapsulation component 15 minutes after culturing under a phase contrast microscope in Experimental Example 9.

FIGS. 16(a) and 16(b) are microscopic images showing the results obtained by observing a cell encapsulation component 1 hour after culturing under a phase contrast microscope in Experimental Example 9.

FIGS. 17(a) to 17(d) are microscopic images showing the results obtained by observing a cell encapsulation component 3 days after culturing under a phase contrast microscope in Experimental Example 9.

FIGS. 18(a) to 18(d) are microscopic images showing the results obtained by observing a cell encapsulation component 7 days after culturing under a phase contrast microscope in Experimental Example 9.

FIGS. 19(a) to 19(n) are images showing procedures of Experimental Example 10.

FIGS. 20(a) to 20(i) are images showing procedures of Experimental Example 10.

FIGS. 21(a) to 21(i) are images of a tubular vitrigel produced in Experimental Example 10.

### DESCRIPTION OF EMBODIMENTS

Embodiments of the present invention will be described below in detail with reference to the drawings in some cases. Here, in the drawings, the same or corresponding parts are denoted with the same or corresponding reference numerals, and redundant descriptions are omitted. Here, the dimensional ratios in the drawings may be exaggerated for explanation, and do not necessarily match the actual dimensional ratios.

### [Tubular Vitrigel]

In one embodiment, the present invention provides a tubular vitrigel composed of a laminate in which a plurality of plate-like vitrigels each having a through-hole are laminated in a thickness direction so that the through-holes are continuous.

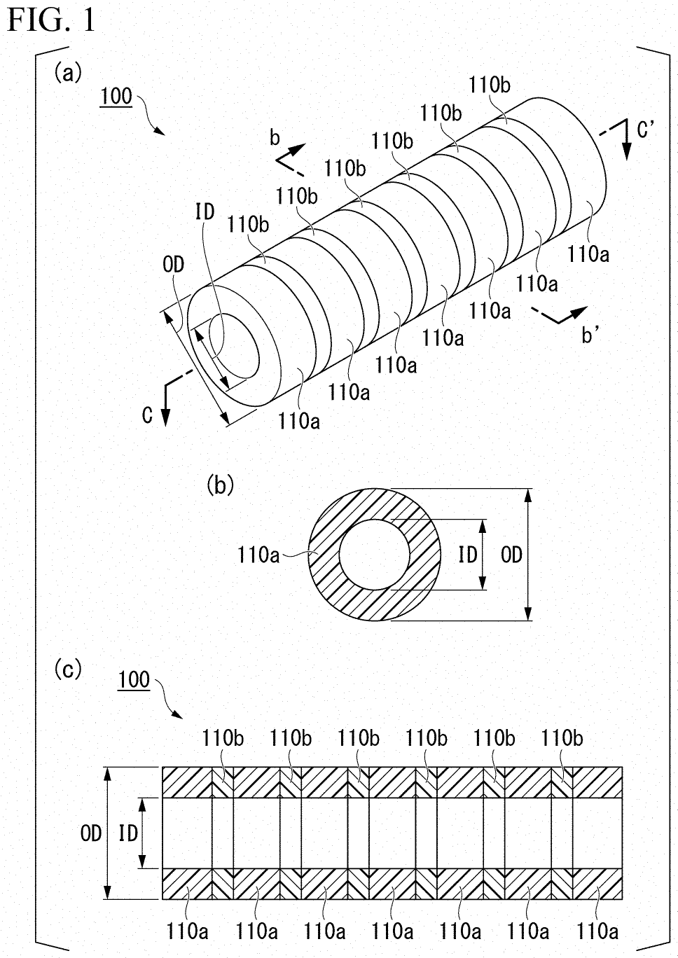

FIGS. 1(a) to 1(c) are schematic views illustrating a structure of an example of a tubular vitrigel of the present embodiment. FIG. 1(a) is a perspective view of the tubular vitrigel of the present embodiment, FIG. 1(b) is a cross-sectional view taken along the arrow line b-b′ in FIG. 1(a), and FIG. 1(c) is a cross-sectional view taken along the arrow line c-c′ in FIG. 1(a). As shown in FIGS. 1(a) to 1(c), a tubular vitrigel 100 is composed of a laminate in which a plurality of plate-like vitrigels 110a or 110b each having a through-hole are laminated in a thickness direction so that the through-holes are continuous.

The tubular vitrigel of the present embodiment can be produced, for example, by a production method to be described below. The plate-like vitrigel 110b is obtained when a sol used for adhering the plate-like vitrigel 110a to another is gelled to finally become a vitrigel in the production method to be described below. Here, in the examples of FIGS. 1(a) to 1(c), the plate-like vitrigel 110a is exposed on the side surface of the tubular vitrigel 100. However, the plate-like vitrigel 110b may cover the side surface of the tubular vitrigel 100, and the plate-like vitrigel 110a may not be exposed on the side surface of the tubular vitrigel 100.

In this specification, assuming a circle having the same area as the narrowest cross-sectional area within a cross-sectional area of the lumen on the plane perpendicular to the tubular vitrigel in the axis direction, the inner diameter of the tubular vitrigel is the diameter of the circle. Here, the tubular vitrigel may be dried component or a hydrated component.

In addition, in this specification, assuming a circle having the same area as the widest cross-sectional area within the cross-sectional area of the outer edge on the plane perpendicular to the tubular vitrigel in the axis direction, the outer diameter of the tubular vitrigel is the diameter of the circle. Here, the tubular vitrigel may be dried component or a hydrated component.

The tubular vitrigel of the present embodiment can have a smaller inner diameter ID than a conventional tubular vitrigel. For example, the inner diameter ID of the tubular vitrigel can be about 5 μm. There is no upper limit on the inner diameter of the tubular vitrigel, which can be, for example, about 10 m, 1 m (100 cm), or 10 cm. In addition, it is easy to make an inner diameter of less than 4 mm, which has been difficult to produce in the related art.

Therefore, the tubular vitrigel of the present embodiment can be used for, for example, small-diameter artificial blood vessels and hollow fibers. As will be described below, a small number of cells can also be retained in the lumen of the hollow fibers. In addition, the tubular vitrigel of the present embodiment can be applied to thicker artificial blood vessels, an artificial ureter, an artificial trachea, an artificial esophagus, an artificial intestinal tract, and the like. In addition, it can be used as an outer cylinder for attaching amputated fingers, arms, and legs.

In the tubular vitrigel of the present embodiment, the size of the outer diameter OD of the tubular vitrigel is not particularly limited, and may be, for example, about 10 m, 1 m (100 cm), or 10 cm, and in consideration of handling properties, it may be, for example, about 0.1 mm to 10 cm. In addition, the length of the tubular vitrigel of the present embodiment in the axis direction is not particularly limited, and can be appropriately set, for example, in a range of 3 mm to 10 m.

In addition, in the examples of FIGS. 1(a) to 1(c), the cross-sectional shape of the lumen on the plane perpendicular to the tubular vitrigel in the axis direction is a circle, but the cross-sectional shape is not limited thereto, and examples thereof include polygons such as a triangle, a quadrangle (including a square, a rectangle, and a trapezoid), a pentagon, a hexagon, a heptagon, and an octagon; substantially a circle, an ellipse, substantially an ellipse, a semicircle, a fan shape, and the like. In addition, in one tubular vitrigel, the cross-sectional shape of the lumen on the plane perpendicular to the axis direction may be constant or may change on the way.

In addition, in the examples of FIGS. 1(a) to 1(c), the cross-sectional shape of the outer edge on the plane perpendicular to the tubular vitrigel in the axis direction is a circle, but the cross-sectional shape of the outer edge is not limited thereto, and examples thereof include polygons such as a triangle, a quadrangle (including a square, a rectangle, and a trapezoid), a pentagon, a hexagon, a heptagon, and an octagon; substantially a circle, an ellipse, substantially an ellipse, a semicircle, a fan shape, and the like. In addition, in one tubular vitrigel, the cross-sectional shape of the outer edge on the plane perpendicular to the axis direction may be constant or may change on the way.

The tubular vitrigel of the present embodiment can be made of a hydrogel having high strength such as native collagen, or can be made of a hydrogel such as atelocollagen which is inferior in strength to native collagen.

In this specification, “sol” means that dispersoid colloidal particles (particle size of about 1 to several hundred nm) using a liquid as a dispersion medium are particularly composed of a polymer compound. More specific sols include, for example, an aqueous solution containing a natural product polymer compound or a synthetic polymer compound. Therefore, when these polymer compounds have a network structure due to chemical bonds, they are transferred to a “hydrogel,” which is a semi-solid substance having a large amount of water in its network. That is, the “hydrogel” is a sol that is gelled. More specific examples of hydrogels include those obtained by introducing crosslinks into artificial materials including a natural product polymer compound or a synthetic polymer compound and performing gelation.

In addition, in this specification, the plate-like vitrigel can also be referred to as a membrane-like vitrigel, a sheet-like vitrigel or the like. The thickness of the plate-like vitrigel of the present embodiment can be appropriately set as necessary, and may be, for example, 0.1 μm to 5 mm, for example, 2 m to 1 mm, or for example, 20 μm to 400 μm.

Examples of the sol that is the material of the tubular vitrigel of the present embodiment include natural polymer compounds such as gelling extracellular matrix-derived components, fibrin, agar, agarose, and cellulose, and synthetic polymer compounds such as polyacrylamide, polyvinyl alcohol, polyethylene oxide, and poly(II-hydroxyethyl methacrylate)/polycaprolactone.

Examples of the above gelling extracellular matrix-derived components include collagens (type I, type II, type III, type V, type XI, etc.), basement membrane components (product name “matrigel”) reconstituted from mouse EHS tumor extracts (including type IV collagen, laminin, heparan sulfate proteoglycans, etc.), glycosaminoglycans, hyaluronic acid, proteoglycans, gelatin, and the like, but the present invention is not limited thereto. A desired hydrogel can be produced by selecting components such as salts that are optimal for each gelation, their concentrations, pH, and the like. Among these, as the sol, a gelling extracellular matrix-derived component is preferable, and collagen is more preferable. In addition, among collagens, native collagen or atelocollagen is still more preferable.

As described above, the term “vitrigel” refers to a gel in a stable state obtained by rehydrating a conventional hydrogel that has been vitrified by drying, and has been named a “vitrigel (registered trademark)” by the inventor.

If the period of the vitrification process, that is, the process in which free water in the hydrogel is completely removed and partial removal of bound water then proceeds, is longer, a vitrigel having excellent transparency and strength can be obtained when rehydrated. Here, as necessary, a vitrigel obtained by rehydration after drying for a short time can be washed with phosphate-buffered saline (PBS) or the like and vitrified again.

Regarding a drying method, for example, various methods such as air drying, drying in a closed container (circulating air in the container and constantly supplying dry air), and drying in an environment in which silica gel is provided, can be used. Examples of air drying methods include drying in a constant temperature and humidity device kept sterile at 10° C. and a relative humidity of 40% for about 2 days, drying in a sterile clean bench for about 24 hours at room temperature, and the like.

In addition, in this specification, the hydrogel dried component that has not been rehydrated after drying may be simply referred to as a “dried component of hydrogel.” Then, the gel obtained by hydrating the hydrogel after drying is distinguishably represented as a “vitrigel.” In this specification, the dried component obtained by drying the vitrigel (vitrigel dried component), and the rehydrate of the vitrigel dried component obtained by additionally rehydrating the vitrigel dried component are also called a vitrigel. In addition, in some cases, the hydrogel and the vitrigel may be referred to as hydrogel without distinguishing therebetween.

The tubular vitrigel of the present embodiment may have larger irregularities of the outer surface of the tube than irregularities of the surface (inner surface) of the lumen of the tubular vitrigel.

More specifically, the irregularities of the outer surface of the tubular vitrigel in the axis direction may be larger than the irregularities of the inner surface of the tubular vitrigel in the axis direction. Here, the irregularities of the outer surface may become large, for example, when the outer diameter of each of the plate-like vitrigels 110a and 110b in FIGS. 1(a) to 1(c) varies. On the other hand, when the tubular vitrigel of the present embodiment is produced by a production method to be described below, the irregularities of the inner surface of the tubular vitrigel in the axis direction are small.

### MODIFICATION EXAMPLES

### First Embodiment

In one embodiment, the present invention provides a tubular vitrigel in which a sheet-like vitrigel is additionally wound around the outer circumference of the above tubular vitrigel.

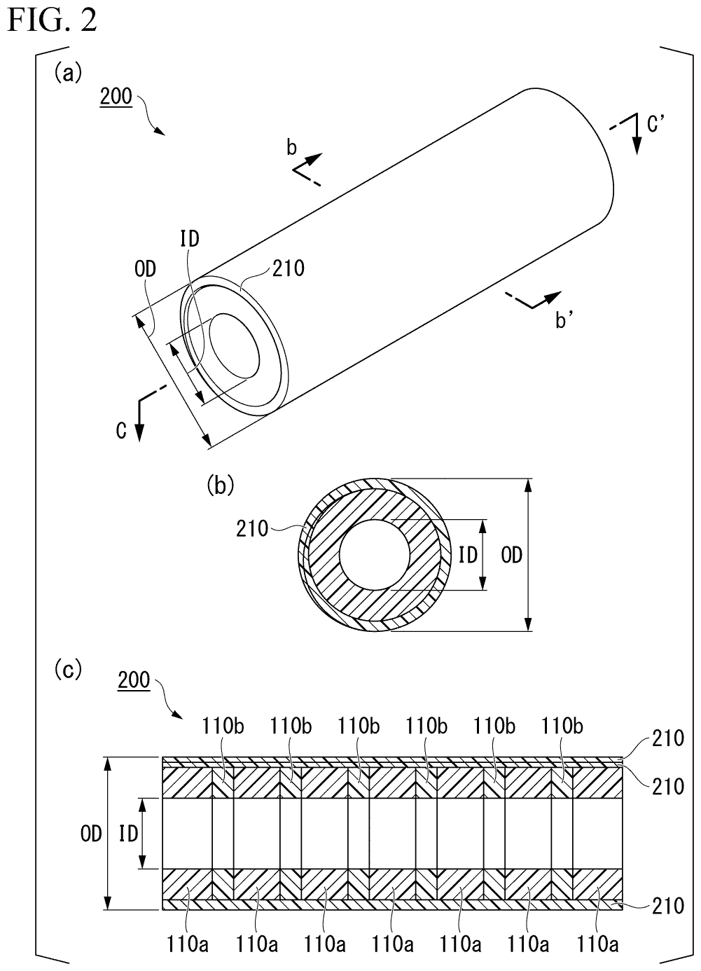

FIGS. 2(a) to 2(c) are schematic views illustrating a structure of an example of a tubular vitrigel of the present embodiment. FIG. 2(a) is a perspective view of the tubular vitrigel of the present embodiment. FIG. 2(b) is a cross-sectional view taken along the arrow line b-b′ in FIG. 2(a), and FIG. 2(c) is a cross-sectional view taken along the arrow line c-c′ in FIG. 2(a). As shown in FIGS. 2(a) to 2(c), a tubular vitrigel 200 has a structure in which a sheet-like vitrigel 210 is additionally wound around the outer circumference of the above tubular vitrigel 100.

The inner diameter of the tubular vitrigel 200 is the same as that of the above tubular vitrigel 100. The outer diameter of the tubular vitrigel 200 is larger than that of the above tubular vitrigel 100 to the extent to which the sheet-like vitrigel 210 is additionally wound.

The thickness of the sheet-like vitrigel can be appropriately set as necessary, and may be, for example, 0.1 μm to 5 mm, for example, 2 μm to 1 mm, or for example, 20 μm to 400 μm.

The strength of the tubular vitrigel 200 is improved because the sheet-like vitrigel 210 is additionally wound. Therefore, for example, it can be used for artificial blood vessels applied to arteries, an artificial trachea, an artificial esophagus, and the like.

The sheet-like vitrigel 210 may have a plurality of through-holes penetrating one side and the other side. Since the sheet-like vitrigel having through-holes tends to have favorable adhesion to an irregular surface, when it is wound around the tubular vitrigel 100, it can adhere better. In addition, also when the tubular vitrigel 200 comes in contact with a living body in a part of the sheet-like vitrigel 210, it can adhere better.

### Second Embodiment

In one embodiment, the present invention provides a tubular vitrigel containing a functional material in at least a part of the above tubular vitrigel. The tubular vitrigel may be a vitrigel in which a sheet-like vitrigel is additionally wound around the outer circumference.

For example, in the tubular vitrigel 100 shown in FIGS. 1(a) to 1(c) or the tubular vitrigel 200 shown in FIGS. 2(a) to 2(c), a part or all of the plate-like vitrigel 110a may contain a functional material, a part or all of the plate-like vitrigel 110b may contain a functional material, and a part or all of the sheet-like vitrigel 210 may contain a functional material.

Examples of functional materials include a support, a magnetic substance, a fluorescent material, a contrast agent, an immunosuppressive agent, an anti-inflammatory agent, and a differentiation-inducing factor.

Examples of supports include a mesh structure. The mesh structure can exhibit functions of reinforcing the strength of the trachea and digestive tract and retaining its shape. Examples of materials of supports include biocompatible organic compounds and biocompatible inorganic compounds, and more specific examples thereof include plastics, metals, ceramics, and the like.

Examples of magnetic substances include magnetic beads. When a part of the tubular vitrigel contains a magnetic substance, for example, it can be moved by applying a magnetic field from the outside of the living body after transplanting it into the living body.

In addition, when a part of the tubular vitrigel contains a fluorescent material, a contrast agent, and the like, for example, after it is transplanted into the living body, the tubular vitrigel can be observed from the outside of the living body through near-infrared fluorescence imaging, Magnetic Resonanse Imaging (MRI), Computed Tomography (CT), or the like.

In addition, when a part of the tubular vitrigel contains an immunosuppressive agent, an anti-inflammatory agent and the like, for example, it is possible to inhibit an inflammatory response when it is transplanted into the living body.

In addition, when a part of the tubular vitrigel contains a differentiation-inducing factor and the like, the tubular vitrigel of the present embodiment can be used for cell differentiation. Details will be described below.

Examples of methods of incorporating a functional material into at least a part of the tubular vitrigel include a method of incorporating a functional material into a plate-like hydrogel used as a material in the tubular vitrigel production process. When a sol as a material for the plate-like hydrogel into which the functional material is incorporated is gelled, it is possible to obtain a plate-like hydrogel containing the functional material.

Alternatively, when the tubular vitrigel is immersed in a liquid containing the functional material, the functional material may be incorporated into the vitrigel constituting the tubular vitrigel.

### [Device for Producing Tubular Vitrigel]

In one embodiment, the present invention provides the above device for producing a tubular vitrigel including a base and a columnar structure that can be fixed to the base. In the production device of the present embodiment, the columnar structure may be fixed to the base.

As will be described below, the outer diameter of the columnar structure corresponds to the inner diameter of the tubular vitrigel.

Therefore, the inner diameter of the tubular vitrigel can be 5 μm or more. When the outer diameter of the columnar structure is small, for example, 1 mm or less, for example, several hundred m or less, for example, several tens m or less, the columnar structure can be said to be a single fibrous structure. The columnar or single fibrous structure may be a linear structure or a structure having at least a partially curved shape.

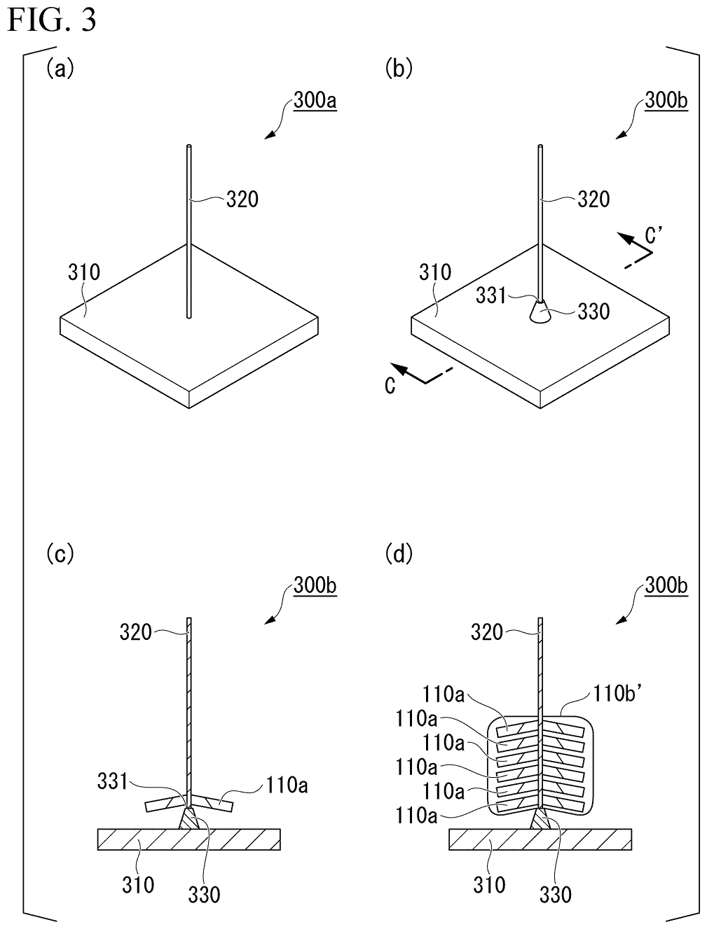

FIG. 3(a) is a perspective view showing an example of a production device of the present embodiment. As shown in FIG. 3(a), a production device 300a includes a base 310, and a columnar or single fibrous structure 320 that can be fixed to the base 310.

The material of the base 310 is not particularly limited, and examples thereof include a resin such as a silicone resin. The material of the columnar or single fibrous structure 320 is not particularly limited, and examples thereof include metals, resins, glass, and biological substances. Examples of metals include stainless steel. That is, as the columnar or single fibrous structure 320, rods, tubes, single fibers, and the like formed of the above materials can be used.

As will be described below, when plate-like hydrogels having through-holes are penetrated and laminated on the columnar or single fibrous structure 320 of the production device 300a of the present embodiment, the above tubular vitrigel can be produced.

Therefore, the outer diameter of the columnar or single fibrous structure 320 corresponds to the inner diameter of the tubular vitrigel. As described above, the inner diameter of the tubular vitrigel can be 5 μm or more.

Therefore, the outer diameter of the columnar or single fibrous structure 320 may be 5 μm or more. Examples of the upper limit value include 10 m, 1 m (100 cm), and 10 cm.

As will be described below in examples, the production device of the present embodiment may further include a pedestal having a larger outer diameter than the columnar or single fibrous structure 320 at the foundation (a part close to the base 310) of the columnar or single fibrous structure 320.

FIG. 3(b) is a perspective view showing a production device 300b further including a pedestal 330. In the production device 300b, the material of the pedestal 330 is not particularly limited, and examples thereof include a resin, glass, and a metal.

The outer diameter of an end 331 on the side opposite to the base 310 of the pedestal 330 is preferably smaller than the outer diameter OD of the tubular hydrogel to be produced. Here, assuming a circle having the same area as the cross-sectional area on the plane perpendicular to the columnar or single fibrous structure 320 of the end 331 of the pedestal 330, the outer diameter of the end 331 of the pedestal 330 is the diameter of the circle.

FIG. 3(c) is a cross-sectional view taken along the line c-c′ in FIG. 3(b), and shows a state in which the plate-like hydrogel 110a having a through-hole is penetrated into the columnar or single fibrous structure 320 of the production device 300b and is in contact with the pedestal 330.

As shown in FIG. 3(c), since the outer diameter of the end 331 of the pedestal 330 is smaller than the outer diameter OD (the outer diameter OD of the plate-like hydrogel 110a) of the tubular hydrogel to be produced, the outer edge part of the plate-like hydrogel 110a is slightly closer to the base 310 due to gravity. Then, when the plate-like hydrogel 110a is additionally penetrated into the columnar or single fibrous structure 320 and laminated, the outer edge parts of the plate-like hydrogels 110a that are close to the base 310 are laminated. The inventors have found that the tubular hydrogel produced by the production device 300b has better adhesion strength between the plate-like hydrogels 110a than the tubular hydrogel produced by the production device 300a.

FIG. 3(d) is a cross-sectional view showing a state in which a plurality of plate-like hydrogels 110a are penetrated into the columnar or single fibrous structure 320 of the production device 300b and laminated. Here, FIG. 3(d) shows a state in which a sol 110b′ is applied to the plate-like hydrogel 110a. The sol 110b′ will be described below.

### Modification Example

The shape of the end on the side opposite to the end 331 of the pedestal 330 is not particularly limited. For example, the production device 300b may be self-supported with only the pedestal 330. In this case, it can be said that the pedestal 330 also serves as the base 310.

### [Method of Producing Tubular Vitrigel]

In one embodiment, the present invention is a method of producing a tubular vitrigel, and provides a producing method including a process (a) in which, on the columnar or single fibrous structure of the above production device, (i) after a sol is applied to surfaces of a plurality of plate-like vitrigels each having a through-hole, the vitrigels are laminated through the through-holes, or (ii) after a plurality of plate-like vitrigels each having a through-hole are laminated through the through-holes, a sol is applied to surfaces, to obtain a plate-like vitrigel laminate having a surface coated with the sol, a process (b) in which the sol is gelled to obtain a tubular hydrogel in which plate-like vitrigels constituting the laminate are connected, a process (c) in which the tubular hydrogel is dried to obtain the tubular hydrogel as a dried component, and a process (d) in which the tubular hydrogel dried component is rehydrated and removed from the columnar or single fibrous structure to obtain a tubular vitrigel. Hereinafter, the processes will be described.

### (Process (a))

In the process (a), (i) after a sol is applied to surfaces of a plurality of plate-like vitrigels each having a through-hole, the through-holes of the plate-like vitrigels penetrate through the columnar or single fibrous structure of the production device and the vitrigels are laminated to obtain a plate-like vitrigel laminate, or (ii) after a plurality of plate-like vitrigels each having a through-hole are laminated on the columnar or single fibrous structure of the production device through the through-holes of the plate-like vitrigels, a sol is applied to surfaces to obtain a plate-like vitrigel laminate having a surface coated with a sol.

FIG. 3(d) is a cross-sectional view showing a state in which a plurality of plate-like hydrogels 110a are penetrated into the columnar or single fibrous structure 320 of the production device 300b and laminated. FIG. 3(d) shows a state in which the sol 110b′ is applied to the plate-like hydrogel 110a. Here, the plate-like hydrogel 110a may be a plate-like vitrigel that has been subjected to the vitrification process.

When the sol 110b′ is a collagen sol, the collagen sol has an optimal salt concentration, and those prepared using, for example, a normal saline, phosphate-buffered saline (PBS), a Hank's Balanced Salt Solution (HBSS), a basic culture medium, a serum-free culture medium, a serum-containing culture medium, or the like, can be used. In addition, the pH of the solution during collagen gelation can be, for example, 6 to 8. In addition, the collagen sol may be prepared at, for example, about 4° C.

In particular, when the serum-free culture medium is used, it is possible to prevent substances not suitable for application to a living body (for example, an antigen, a pathogenic factor, etc.), which are contained in other animal serum components, from being incorporated into the hydrogel. Therefore, the hydrogel obtained using the serum-free culture medium is suitably used for medical applications.

In addition, the concentration of the collagen sol for producing a hydrogel is preferably 0.1 to 1.0 mass % and more preferably 0.2 to 0.6 mass %. When the concentration of the collagen sol is the lower limit value or more, gelation is not too weak, and when the concentration of the collagen sol is the upper limit value or less, a hydrogel made of a uniform collagen gel can be obtained.

The above functional material may be incorporated into at least a part of the plate-like hydrogel 110a or the sol 110b′. Thereby, a tubular vitrigel containing a functional material in at least a part can be produced.

### (Process (b))

In the process (b), the sol 110b′ is gelled to obtain a tubular hydrogel in which plate-like vitrigels constituting the laminate are connected. The temperature at which the sol 110b′ is kept can be appropriately adjusted according to the type of sol used. For example, when the sol is a collagen sol, heat retention during gelation can be performed at a temperature lower than a denaturation temperature of collagen depending on the animal species of collagen used, and generally, the temperature can be kept at 20° C. or higher and 37° C. or lower, and thus gelation can be performed for a few minutes to a few hours.

### (Process (c))

In the process (c), the tubular hydrogel obtained in the process (b) is dried to obtain the tubular hydrogel as a dried component. The drying method is the same as that described above, and for example, various methods such as air drying, drying in a closed container, and drying in an environment in which silica gel is provided can be used.

In one embodiment, ultraviolet rays may be irradiated to the tubular hydrogel dried component obtained in this process. Ultraviolet rays can be irradiated using a known ultraviolet ray irradiation device. Regarding energy for irradiating ultraviolet rays to the tubular hydrogel dried component, the total irradiation amount per unit area is preferably 0.1 mJ/cm² to 6,000 mJ/cm², more preferably 10 mJ/cm² to 4,000 mJ/cm², and still more preferably 100 mJ/cm² to 3,000 mJ/cm². When the total irradiation amount is within the above range, the transparency and strength of the tubular vitrigel obtained by subsequent rehydration can be particularly preferable.

In addition, irradiation of ultraviolet rays to the tubular hydrogel dried component may be repeated a plurality of times.

When ultraviolet rays are repeatedly irradiated to the tubular hydrogel dried component, after first irradiation of ultraviolet rays is performed, a process of rehydrating and revitrifying the tubular hydrogel dried component that has been subjected to the ultraviolet ray irradiation treatment is performed, and ultraviolet rays may be then irradiated to the dried component of the tubular vitrigel after the second and subsequent re-vitrification.

When a total irradiation amount of ultraviolet rays per unit area is the same, if ultraviolet rays are repeatedly irradiated to the tubular hydrogel dried component a plurality of times in a divided manner, it is possible to further improve the transparency and strength of the tubular vitrigel obtained in the subsequent rehydration process. In addition, a larger number of divisions is more preferable. For example, when the total amount of ultraviolet rays per unit area irradiated to the tubular hydrogel dried component is in a range of 1,000 mJ/cm² to 4,000 mJ/cm², the number of irradiations within the range may be 2 to 10 or 2 to 6.

In addition, when ultraviolet rays are repeatedly irradiated to the tubular hydrogel dried component, for example, the tubular hydrogel dried component is rotated, and ultraviolet rays are irradiated while changing the surface to which ultraviolet rays are irradiated, a total irradiation amount per unit area of all of the irradiation surfaces may be used as a total amount of ultraviolet rays irradiated to the tubular hydrogel dried component.

When ultraviolet rays are irradiated to the tubular hydrogel dried component, the strength and transparency of the tubular vitrigel obtained in the subsequent rehydration process are improved. It is thought that this is because polymer compounds in the tubular vitrigel are crosslinked with ultraviolet rays. That is, according to the operation, high transparency and strength can be imparted to the tubular vitrigel.

In addition, the obtained tubular hydrogel dried component that has been subjected to an ultraviolet ray irradiation treatment may be rehydrated with PBS, a culture medium or the like to obtain a tubular vitrigel. In addition, the obtained tubular vitrigel may be dried and revitrified to obtain the tubular vitrigel as a dried component.

### (Process (d))

In the process (d), the tubular hydrogel dried component obtained in the process (c) is rehydrated and removed from the columnar or single fibrous structure of the production device to obtain a tubular vitrigel. The obtained tubular vitrigel may be repeatedly subjected to the drying process and the hydrating process once or a plurality of times.

### MODIFICATION EXAMPLES

### First Embodiment

As described above, a sheet-like vitrigel may be additionally wound around the outer circumference of the tubular vitrigel. In this case, after the process (a), and before the process (b), a process in which a sheet-like hydrogel coated with a sol on its surface is wound around a plate-like vitrigel laminate may be further provided.

Alternatively, after the process (b), and before the process (c), a process in which a sheet-like hydrogel coated with a sol on its surface is wound around the tubular hydrogel and the sol is gelled to obtain a tubular hydrogel may be further provided.

Alternatively, after the process (c) and before the process (d), a process in which a sheet-like hydrogel coated with a sol on its surface is wound around the tubular hydrogel dried component, the sol is gelled to obtain a tubular hydrogel, and the tubular hydrogel is dried to obtain the tubular hydrogel as a dried component may be further provided.

In this case, the sheet-like hydrogel may be wound to obtain the tubular hydrogel as a dried component after the process (c) and before the above ultraviolet ray irradiation, or after the above ultraviolet ray irradiation and before the process (d).

### Second Embodiment

In one embodiment, the present invention provides a tubular vitrigel composed of a wound sheet-like vitrigel.

The tubular vitrigel of the present embodiment can be produced, for example, by a production method including a process in which a sol is applied to one surface of a sheet-like vitrigel, a process in which the one surface of the sheet-like vitrigel is brought into contact with a columnar structure and wound therearound, a process in which the sol is gelled to obtain a tubular hydrogel, a process in which the tubular hydrogel is dried to obtain the tubular hydrogel as a dried component, and a process in which the tubular hydrogel dried component is rehydrated and removed from the columnar structure to obtain a tubular vitrigel.

As described above, in the production method described in Patent Literature 1, it is difficult to produce a tubular vitrigel having an inner diameter of less than 4 mm. In addition, in the production method described in Patent Literature 1, it is not possible to produce a tubular vitrigel using a hydrogel such as atelocollagen, which is inferior in strength to native collagen.

On the other hand, as will be described below in examples, according to the production method of the present embodiment, it is also possible to produce a tubular vitrigel having an inner diameter of less than 4 mm. In addition, according to the production method of the present embodiment, it is also possible to produce a tubular vitrigel having an inner diameter of 4 mm or more.

In addition, according to the production method of the present embodiment, since it is not necessary to set an angle formed in the long axis direction of the shape of the hydrogel with respect to a horizontal direction, it is also possible to produce a tubular vitrigel using a sheet-like vitrigel such as atelocollagen, which is inferior in strength to native collagen.

Regarding the columnar structure, for example, rods and tubes formed of stainless steel, glass, a resin or the like can be used. The outer diameter of the columnar structure corresponds to the inner diameter of the tubular vitrigel.

Therefore, for example, when the inner diameter of the tubular vitrigel is 1 to 4 mm, the outer diameter of the columnar structure can be 1 to 4 mm.

### [Artificial Tubular Tissue]

In one embodiment, the present invention provides an artificial tubular tissue composed of the above tubular vitrigel. Examples of artificial tubular tissues include artificial blood vessels, an artificial ureter, an artificial trachea, an artificial digestive tract, and the like. In addition, the above tubular vitrigel can be used as an outer cylinder for attaching amputated fingers, arms, and legs. In this specification, such an outer cylinder is also included in the artificial tubular tissues.

As described above, the artificial tubular tissue of the present embodiment can have an inner diameter of 5 μm to several cm or more. Examples of the upper limit value include 10 m, 1 m (100 cm), 10 cm, and the like. Therefore, it can be applied not only to small-diameter artificial blood vessels but also thicker artificial blood vessels, an artificial ureter, an artificial trachea, an artificial esophagus, an artificial intestinal tract, and the like.

In addition, the strength of the artificial blood vessel of the present embodiment can be improved by, for example, a method such as additionally winding a sheet-like vitrigel around the tubular vitrigel. Therefore, it can be applied to arteries.

In addition, as described above, the artificial blood vessel of the present embodiment may contain, for example, a support, a magnetic substance, a fluorescent material, a contrast agent, an immunosuppressive agent, an anti-inflammatory agent, a differentiation-inducing factor, and the like.

### [Cell Encapsulation Device and Cell Encapsulation Component]

In one embodiment, the present invention provides a cell encapsulation device composed of the above tubular vitrigel.

The inventors have found that, when the inner diameter of the tubular vitrigel is particularly 1.5 mm or less, a liquid tends to be retained inside the tubular vitrigel.

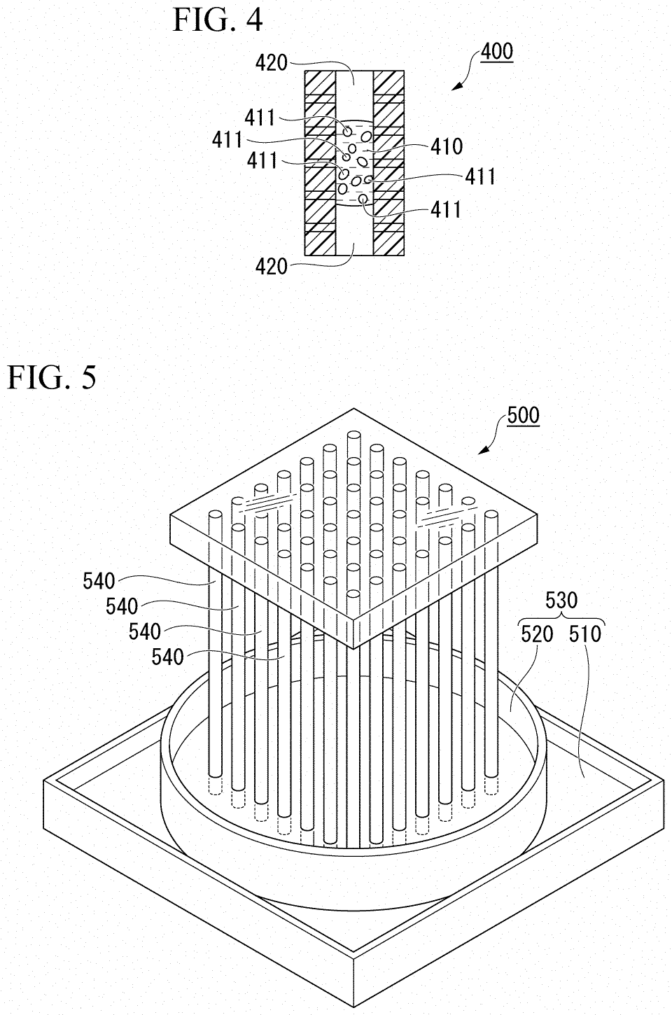

FIG. 4 is a schematic cross-sectional view showing a structure of a cell encapsulation component 400 in which a liquid (a cell suspension 410) is retained inside the tubular vitrigel 100. In FIG. 4, the tubular vitrigel is retained so that the long axis direction is the vertical direction. Air is present above and below the cell suspension 410. According to the cell encapsulation device of the present embodiment, the cell suspension can be stably retained and cells can be encapsulated in the state shown in FIG. 4.

The cell suspension 410 can be retained in the cell encapsulation device, for example, as follows. First, the cell encapsulation device is held so that the long axis direction is the vertical direction. Next, sucking is performed from the upper end of the cell encapsulation device to suck up the cell suspension 410. Next, additional sucking is performed from the upper end of the cell encapsulation device to suck up air 420. As a result, it is possible to obtain a cell encapsulation component in which a liquid containing cells is retained in the central part of the cell encapsulation device in the long axis direction, and air is present at both end parts in the long axis direction.

Alternatively, as will be described below in examples, when the cell suspension 410 is injected into the central part of the cell encapsulation device in the long axis direction using a syringe with a needle or the like, it is possible to obtain a cell encapsulation component in which a liquid containing cells is retained in the central part of the cell encapsulation device in the long axis direction, and air is present at both end parts in the long axis direction.

In the cell encapsulation component, the air 420 functions as a plug that encapsulates cells. In addition, as will be described below in examples, when a sol is injected into both ends of the cell encapsulation component and gelled, a stronger plug can be formed.

For example, therapeutic cells can be encapsulated in the cell encapsulation component of the present embodiment and transplanted into a living body. The therapeutic cells are not particularly limited, and examples thereof include pancreatic islet cells and dopamine-producing cells.

In addition, as described above, the cell encapsulation component of the present embodiment may contain functional materials, for example, a support, a magnetic substance, a fluorescent material, a contrast agent, an immunosuppressive agent, an anti-inflammatory agent, a differentiation-inducing factor, and the like. The functional materials are the same as those described above. Therefore, it is possible to have functions such as moving by applying a magnetic field to the cell encapsulation component from the outside of the living body, observing the cell encapsulation component from the outside of the living body, and inhibiting an inflammatory response and the like when the cell encapsulation component is transplanted into the living body.

According to the cell encapsulation component of the present embodiment, functional materials can be contained in the cell encapsulation device. Therefore, for example, compared with when a functional material such as a magnetic substance is introduced into cells, it is possible to reduce toxicity to the cells.

In addition, in the cell encapsulation component of the present embodiment, for example, pluripotent stem cells such as embryonic stem cells (ES cells) and induced pluripotent stem cells (iPS cells) can be encapsulated and differentiated into desired cells by the action of the differentiation-inducing factor.

The differentiation-inducing factor may be contained in the tubular vitrigel in advance and supplied from the tubular vitrigel in a sustained release manner and act on cells. Alternatively, when the cell encapsulation component of the present embodiment is immersed in a culture medium containing the differentiation-inducing factor, the differentiation-inducing factor in the culture medium may permeate the tubular vitrigel and act on the cells.

Here, a plurality of types of differentiation-inducing factors may be contained in the tubular vitrigel in advance and predetermined differentiation-inducing factors may be sequentially supplied at predetermined timings. Alternatively, a plurality of culture media containing different differentiation-inducing factors may be prepared, and the cell encapsulation component of the present embodiment may be sequentially immersed in a culture medium containing predetermined differentiation-inducing factors at predetermined timings. Thereby, it is possible to differentiate pluripotent stem cells into desired cells.

### EXAMPLES

While the present invention will be described below in more detail with reference to examples, the present invention is not limited to the following examples.

### Production Example 1

A plate-like vitrigel having through-holes was produced using a production device shown in FIG. 5. FIG. 5 is a schematic view showing a structure of a production device used in this production example. As shown in FIG. 5, a production device 500 had a container part 530 having a bottom part 510 and a side part 520, and a plurality of columnar jigs 540 that could be arranged in contact with the bottom part 510. As the columnar jig 540, a cylindrical stainless steel shaft having a diameter of 1 mm was used.

First, the production device was sterilized with 70% ethanol and dried in a clean bench. Next, 16 mL of a serum-free culture medium was dispensed into a 50 mL conical tube on ice. Next, 16 mL of porcine atelocollagen (commercially available from Kanto Chemical Co., Inc., collagen concentration of 1 mass %) was added to prepare a uniform collagen sol. As the serum-free culture medium, a DMEM medium (catalog number “11885-084,” commercially available from Thermo Fisher Scientific) containing 20 mM HEPES (catalog number “15630-080,” commercially available from Thermo Fisher Scientific), 100 unit/mL penicillin, and 100 μg/mL streptomycin (catalog number “15140-148,” commercially available from Thermo Fisher Scientific) was used.

Next, 26.7 mL of a sol was injected into the container part of the production device 500. Next, the production device was left in an incubator set at 37° C., 5% CO₂ for 2 hours for gelation to obtain a hydrogel.

Next, free water in the hydrogel was dehydrated from the gap between the bottom part and the side part of the container and dried from the top surface, and when the height of the hydrogel reached 6 mm, the porous hydrogel was removed from the production device, and dried and vitrified in a constant temperature and humidity device kept sterile at 10° C. and a relative humidity of 40%.

Next, after vitrification, the hydrogel was removed from the constant temperature and humidity device and rehydrated in PBS. In addition, a washing process by rehydration with fresh PBS was repeated, washing was performed a total of three times, re-vitrification was then performed by drying in a constant temperature and humidity device kept sterile at 10° C. and a relative humidity of 40%, and thereby a “plate-like vitrigel dried component having through-holes having a diameter of 1 mm” of Production Example 1 was obtained.

### Experimental Example 1

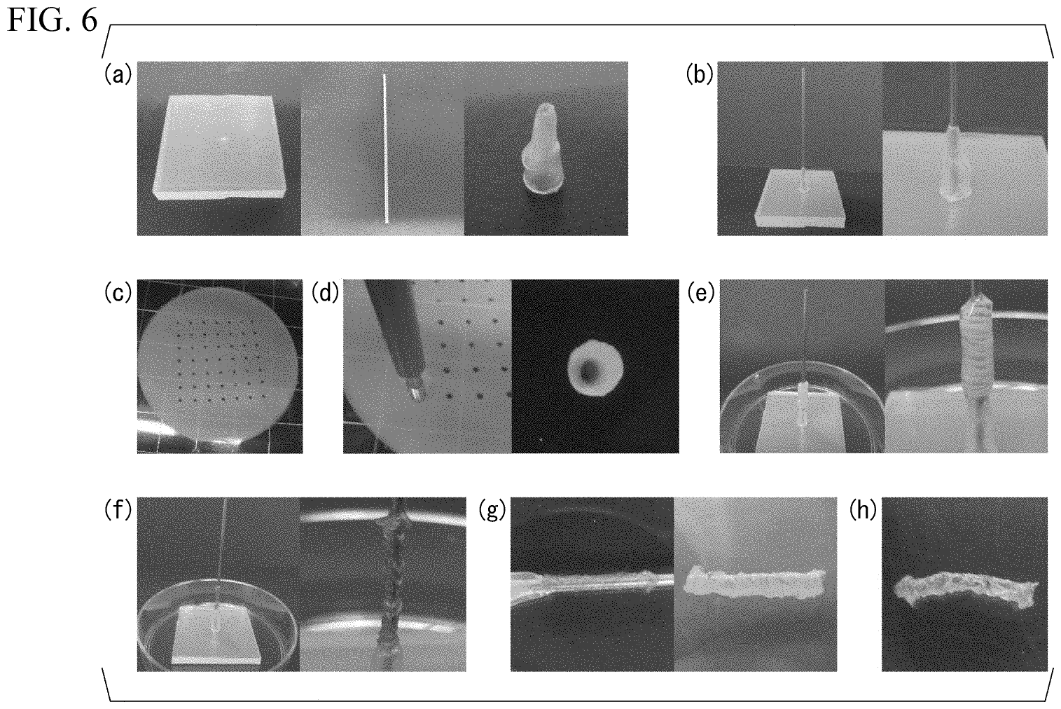

In this Experimental Example, first, a device for producing a tubular vitrigel was produced, and subsequently, a tubular vitrigel was produced. FIGS. 6(a) to 6(h) are images showing procedures of this Experimental Example.

### (Production of Device for Producing Tubular Vitrigel)

As shown in FIG. 6(a), a 5 mm-thick silicon sheet, a stainless steel rod having a diameter 1 mm and a length 5 cm, and a tip of a 1,000 μL micropipette tip were prepared. Next, the silicon sheet was cut into 3×3 cm and used as a base, and a hole having a diameter of 1 mm was formed in the center.

Next, as shown in FIG. 6(b), a stainless steel rod (columnar structure) was erected in the hole formed in the silicon sheet, and a pedestal formed from the tip of the 1,000 μL micropipette tip was placed at the foundation of the stainless steel rod.

### (Production of Tubular Vitrigel)

First, the “plate-like vitrigel dried component having through-holes having a diameter of 1 mm” produced in Production Example 1 was rehydrated in PBS. FIG. 6(c) is an image of the plate-like vitrigel having through-holes having a diameter of 1 mm. Next, as shown in FIG. 6(d), a plurality of plate-like vitrigels each having a through-hole were produced by cutting out the periphery of the through-hole of the plate-like hydrogel using a trepan having a diameter of 2.5 mm.

Next, 2 mL of a serum-free medium and 2 mL of porcine atelocollagen which were the same as those in Production Example 1 were mixed on ice to prepare a uniform collagen sol. Next, the above plate-like vitrigel having a through-hole was immersed in the above collagen sol, and the through-hole of the plate-like vitrigel penetrated through the stainless steel rod of the production device shown in FIG. 6(b). This operation was repeated to obtain a laminate in which a total of 24 plate-like vitrigels were laminated. FIG. 6(e) is an image showing a state in which plate-like vitrigels are laminated.

Next, a small amount of sol around the plate-like vitrigel laminate was removed and gelation was caused in the clean bench at room temperature to obtain a tubular hydrogel. Next, as shown in FIG. 6(f), the tubular hydrogel with accompanying the production device was dried and vitrified in the constant temperature and humidity device kept sterile at 10° C. and a relative humidity of 40% to obtain the tubular hydrogel as a dried component. Next, ultraviolet rays were irradiated to the tubular hydrogel dried component. The irradiation amount of ultraviolet rays was 800 mJ/cm² per direction, and irradiation was performed in four directions rotated 90°.

Next, as shown in FIG. 6(g), the tubular hydrogel dried component was rehydrated in PBS and removed from the stainless steel rod to obtain a tubular vitrigel of the hydrated component. Next, as shown in FIG. 6(h), the obtained tubular vitrigel of the hydrated component was dried again and revitrified in the constant temperature and humidity device kept sterile at 10° C. and a relative humidity of 40% to obtain the tubular vitrigel as a dried component.

### Experimental Example 2

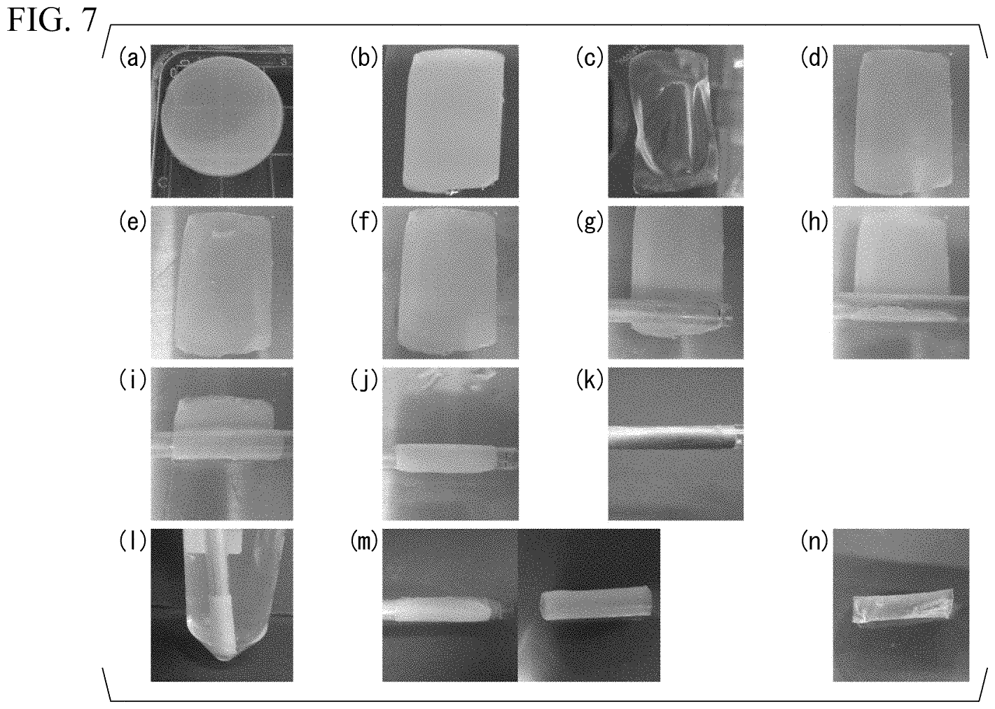

In this Experimental Example, the plate-like vitrigel was wound around a stainless steel tube having an outer diameter of 4.57 mm to produce a tubular vitrigel. FIGS. 7(a) to 7(n) are images showing procedures of this Experimental Example.

The dried component of the atelocollagen vitrigel membrane (collagen amount: 5 mg/cm²) produced in advance was rehydrated in PBS to prepare a plate-like vitrigel. FIG. 7(a) is an image of the plate-like vitrigel. Next, as shown in FIG. 7(b), the rehydrated plate-like vitrigel was cut into a rectangular shape (20 mm×32 mm). Next, as shown in FIG. 7(c), the plate-like vitrigel cut into a rectangular shape was dried again and revitrified, and 200 mJ/cm² of ultraviolet rays were irradiated to only one side. Next, as shown in FIG. 7(d), the plate-like vitrigel to which ultraviolet rays were irradiated was rehydrated in PBS.

Next, 2.5 mL of a serum-free medium and 2.5 mL of porcine atelocollagen which were the same as those in Production Example 1 were mixed on ice to prepare a uniform collagen sol. Next, as shown in FIG. 7(e), 200 μL of the collagen sol was left on the above plate-like vitrigel. Next, as shown in FIG. 7(f), the collagen sol was spread over the entire surface of the plate-like vitrigel.

Next, a polyethylene terephthalate (PET) film having one side treated with silicon was attached to the outer surface of the stainless steel tube having an outer diameter 4.57 mm with a dual-sided tape. In this case, the outer side was used as a silicon-treated surface. Next, as shown in FIGS. 7(g) to 7(i), the plate-like vitrigel was wound around the stainless steel tube.

Next, as shown in FIG. 7(j), gelation was caused in the clean bench at room temperature to obtain a tubular hydrogel. Next, the tubular hydrogel with accompanying the stainless steel tube was dried and vitrified in the constant temperature and humidity device kept sterile at 10° C. and a relative humidity of 40% to obtain the tubular hydrogel as a dried component. Next, as shown in FIG. 7(k), ultraviolet rays were irradiated to the tubular hydrogel dried component. The irradiation amount of ultraviolet rays was 200 mJ/cm² per direction, and irradiation was performed in four directions rotated 90°.

Next, as shown in FIG. 7(l), the tubular hydrogel dried component was rehydrated in PBS. Next, as shown in FIG. 7(m), the sample was removed from the stainless steel tube to obtain a tubular vitrigel of the hydrated component. Next, as shown in FIG. 7(n), the obtained tubular vitrigel of the hydrated component was dried again and revitrified in the constant temperature and humidity device kept sterile at 10° C. and a relative humidity of 40% to obtain the tubular vitrigel as a dried component.

### Experimental Examples 3 and 4

A tubular vitrigel of Experimental Example 3 was produced in the same manner as in Experimental Example 2 except that a stainless steel tube having an outer diameter of 2.41 mm was used. In addition, a tubular vitrigel of Experimental Example 4 was produced in the same manner as in Experimental Example 2 except that a stainless steel tube having an outer diameter of 1.26 mm was used. In the tubular vitrigel of Experimental Example 4, it was difficult to wind the plate-like vitrigel around the stainless steel tube.

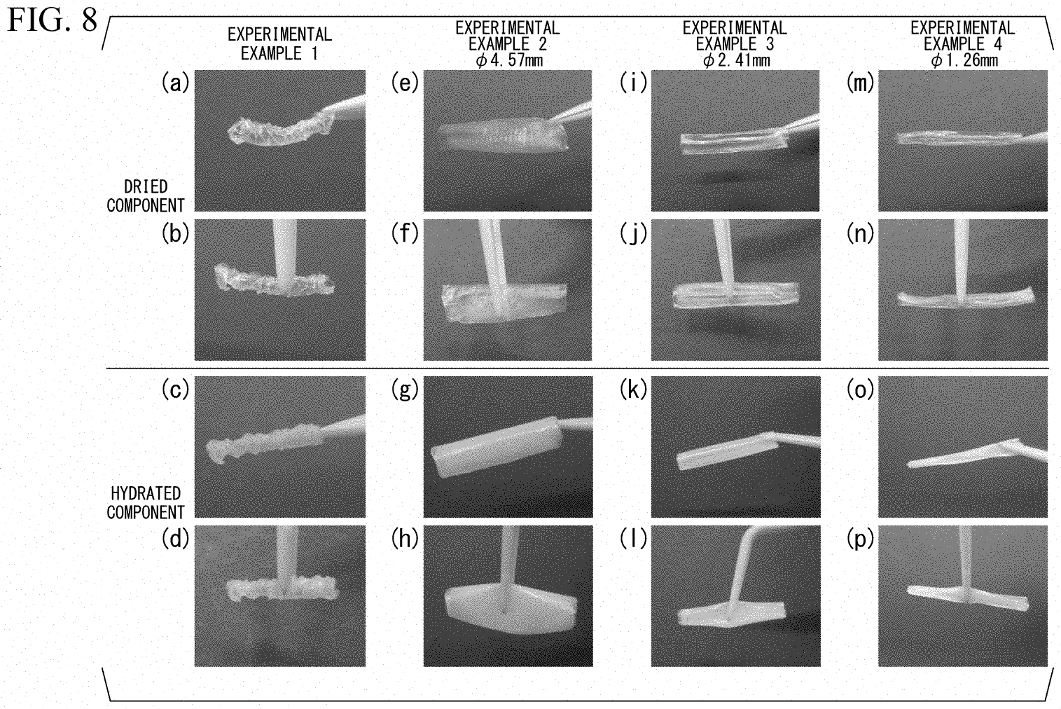

FIGS. 8(a) to (p) are images of the tubular vitrigels of Experimental Examples 1 to 4. FIGS. 8(a) and 8(b) are images of the tubular vitrigel dried component of Experimental Example 1. FIGS. 8(c) and 8(d) are images of the tubular vitrigel of the hydrated component of Experimental Example 1.

FIGS. 8(e) and 8(f) are images of the tubular vitrigel dried component of Experimental Example 2. FIGS. 8(g) and 8(h) are images of the tubular vitrigel of the hydrated component of Experimental Example 2.

FIGS. 8(i) and 8j) are images of the tubular vitrigel dried component of Experimental Example 3. FIGS. 8(k) and 8(l) are images of the tubular vitrigel of the hydrated component of Experimental Example 3.

FIGS. 8(m) and 8(n) are images of the tubular vitrigel dried component of Experimental Example 4. FIGS. 8(o) and 8(p) are images of the tubular vitrigel of the hydrated component of Experimental Example 4.

As a result, it can be clearly understood that the tubular vitrigel of Experimental Example 1 could be easily pinched with tweezers regardless of whether it was in a dried component state or a hydrated component state and had favorable handling properties. On the other hand, the tubular vitrigels of Experimental Examples 2 to 4 tended to be slippery when pinched with tweezers, regardless of they were in a dried component state or a hydrated component state. In addition, when the tube was pinched with tweezers in the direction perpendicular to the axial direction of the tube, the tube tended to be easily crushed.

Based on the above results, it can be clearly understood that the tubular vitrigel of Experimental Example 1 tended to have a smaller inner diameter than the tubular vitrigels of Experimental Examples 2 to 4, and tended to have better handling properties.

### Experimental Example 5

### (Passing Through Tubular Vitrigel)

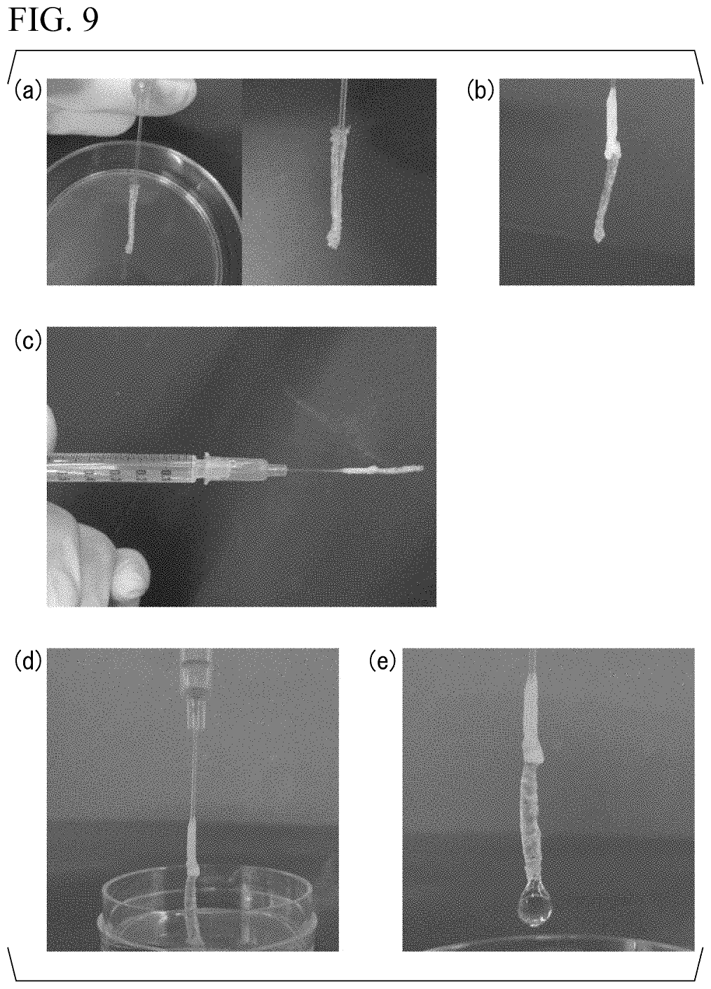

It was confirmed that sterile water could be passed through the tubular vitrigel of Experimental Example 1. FIGS. 9(a) to 9(e) are images showing procedures of this Experimental Example.

First, as shown in FIG. 9(a), an indwelling needle catheter (product number “NIC*26×¾,” a catheter (gauge 26G) and an inner needle (gauge 27G) both commercially available from Nipro Corporation) was inserted into the tubular vitrigel of Experimental Example 1.

Next, as shown in FIG. 9(b), the insertion part of the indwelling needle catheter was fixed with parafilm. Next, as shown in FIG. 9(c), a 1 mL syringe was attached. Next, as shown in FIG. 9(d), sterile water was sucked up with the syringe. Next, sterile water was extruded with the syringe. As a result, as shown in FIG. 9(e), it was confirmed that sterile water could be passed through the tubular vitrigel of Experimental Example 1.

### Experimental Example 6

(Injection of PBS into Tubular Vitrigel)

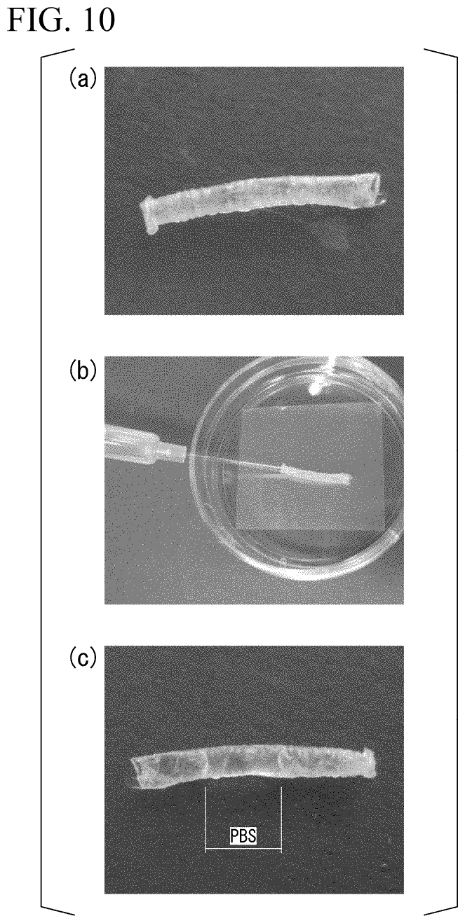

It was confirmed that a liquid could be retained inside the tubular vitrigel of Experimental Example 1. FIGS. 10(a) to 10(c) are images showing procedures of this Experimental Example.

FIG. 10(a) is an image of the tubular vitrigel dried component of Experimental Example 1. As shown in FIG. 10(b), the indwelling needle catheter was inserted into the tubular vitrigel of Experimental Example 1, and PBS was injected into the central part of the tubular vitrigel.

FIG. 10(c) is an image of the tubular vitrigel after injection of PBS. As a result, it was confirmed that a PBS encapsulation component in which PBS was retained in the central part in the long axis direction and air was present at both end parts in the long axis direction was obtained.

### Experimental Example 7

(Injection of Serum-Free Culture Medium into Tubular Vitrigel)

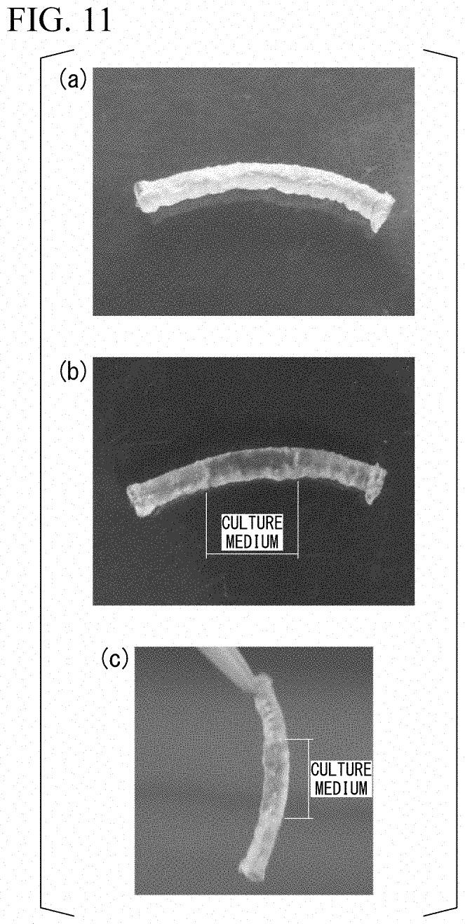

It was confirmed that a liquid could be retained inside the tubular vitrigel of Experimental Example 1. FIGS. 11(a) to 11(c) are images showing procedures of this Experimental Example.

FIG. 11(a) is an image of the tubular vitrigel dried component of Experimental Example 1. As shown in FIG. 11(b), the indwelling needle catheter was inserted into the tubular vitrigel of Experimental Example 1, and a serum-free culture medium was injected into the central part of the tubular vitrigel. As a result, it was confirmed that a serum-free culture medium encapsulation component in which a serum-free culture medium was retained in the central part in the long axis direction and air was present at both end parts in the long axis direction was obtained.

FIG. 11(c) is an image showing a state in which the tubular vitrigel after the serum-free culture medium was injected, was pinched with tweezers and supported in the vertical direction. As a result, it was confirmed that the encapsulated serum-free culture medium did not leak even when supported in the vertical direction.

This result indicates that, in the medical field and the like, the cell suspension could be easily handled with tweezers or the like when encapsulated in the tubular vitrigel. Therefore, it was expected that the tubular vitrigel could be used for transplantation of cells suspended in a culture medium or the like.

### Experimental Example 8

(Injection of Cells into Tubular Vitrigel)

Cells were encapsulated inside the tubular vitrigel of Experimental Example 1.

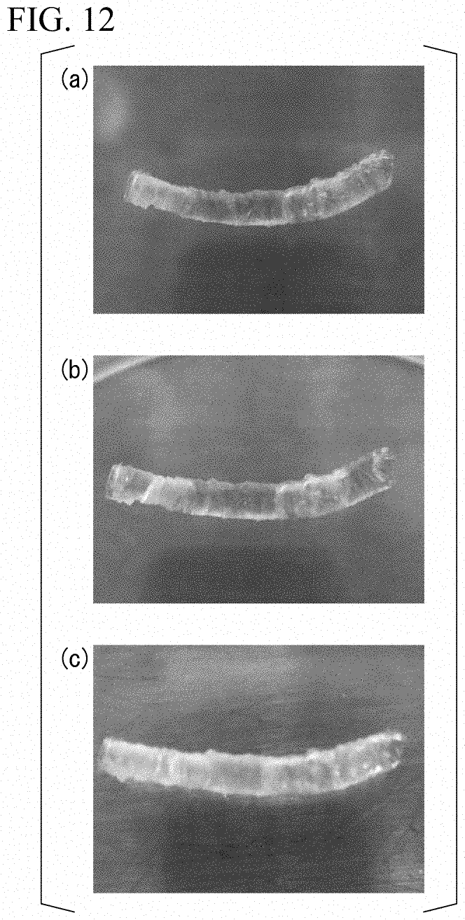

FIGS. 12(a) to 12(c) are images showing procedures of this Experimental Example. As shown in FIG. 12(a), using a gel loading tip (product number “2-4493-06,” commercially available from As One Corporation), 7 μL (1×10⁴ cells) of the suspension of human dermal fibroblasts was injected into the central part of the tubular vitrigel of Experimental Example 1.

Next, as shown in FIG. 12(b), 3 to 4 μL of 0.5% atelocollagen sol was injected into each end of the tubular vitrigel. Next, as shown in FIG. 12(c), the sample was left in a 5% carbon dioxide gas incubator for cell culture at 37° C. for 5 minutes. As a result, the atelocollagen sol was gelled to obtain a cell encapsulation component.

### Experimental Example 9

### (Culture of Cells Using Cell Encapsulation Component)

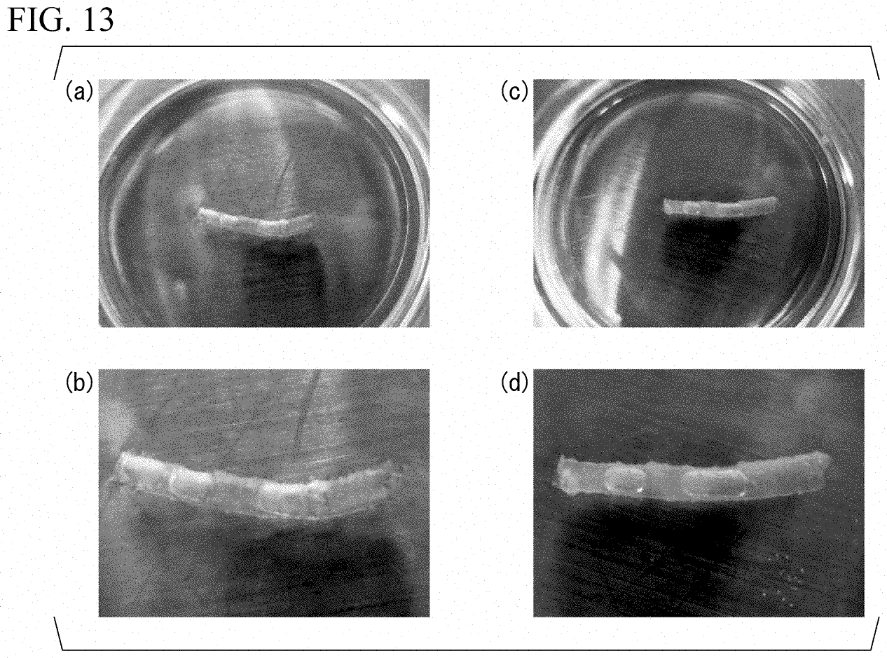

Cells in the cell encapsulation component produced in Experimental Example 8 were cultured. First, the cell encapsulation component floated in the culture medium. FIG. 13(a) is an image showing a state in which the cell encapsulation component floated in the culture medium. In addition, FIG. 13(b) is an enlarged image of FIG. 13(a). In addition, FIG. 13(c) is an image showing a state 5 minutes after the cell encapsulation component floats in the culture medium. In addition, FIG. 13(d) is an enlarged image of FIG. 13(c).

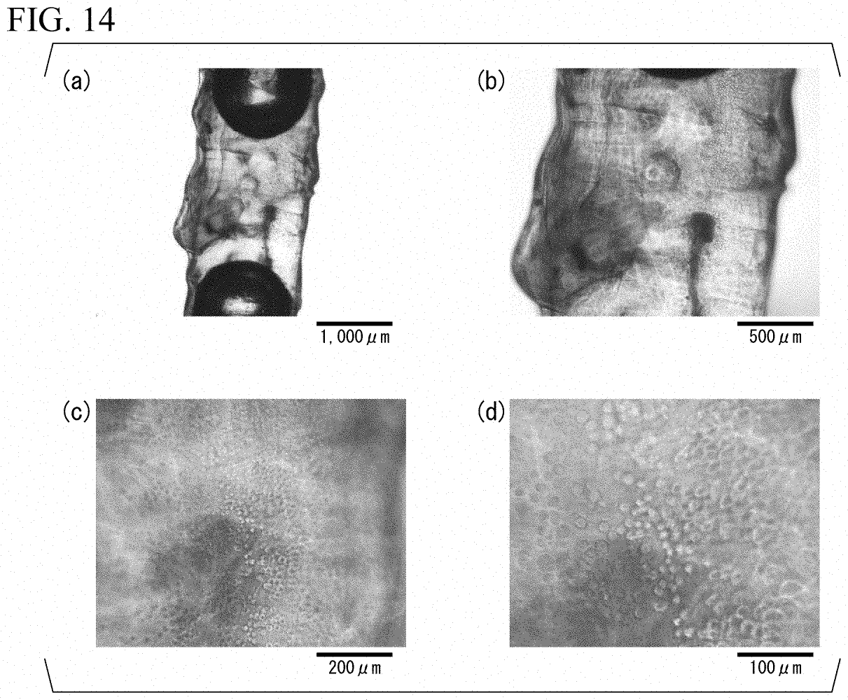

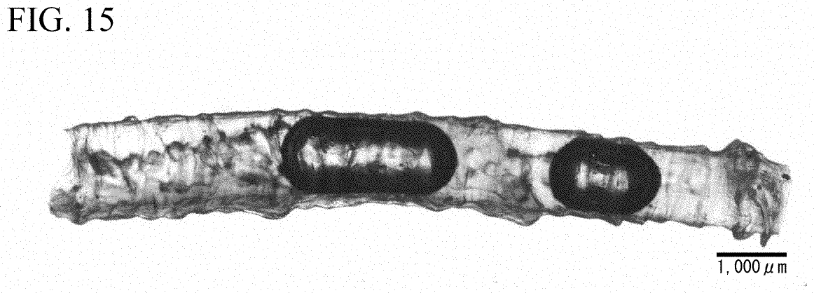

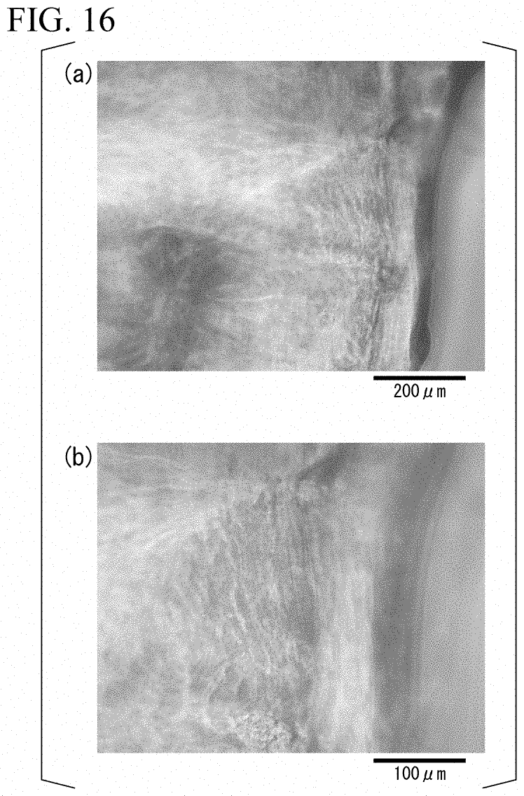

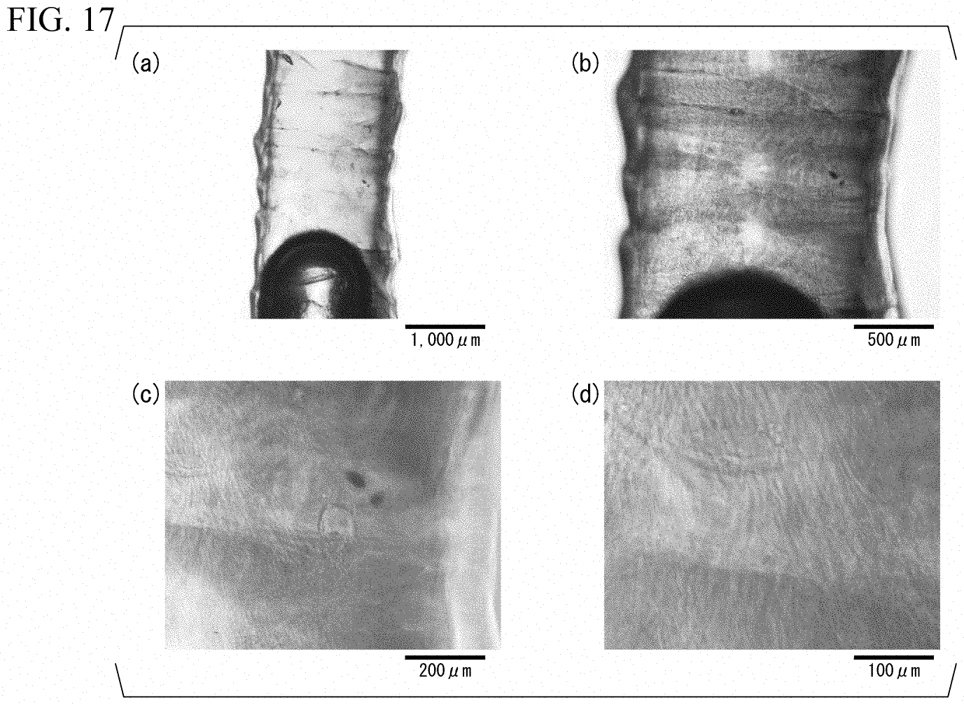

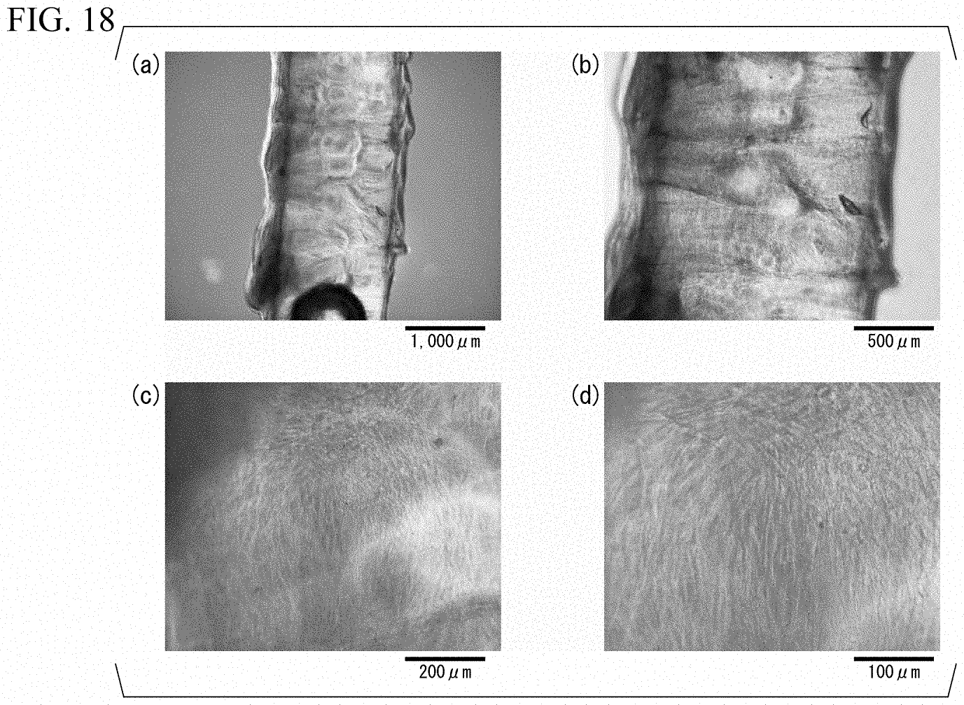

FIGS. 14(a) to 14(d) are microscopic images showing the results obtained by observing the cell encapsulation component 15 minutes after culture under a phase contrast microscope. FIG. 15 is an image showing the result obtained by observing the entire cell encapsulation component 15 minutes after culture under a phase contrast microscope. FIGS. 16(a) and 16(b) are microscopic images showing the results obtained by observing the cell encapsulation component 1 hour after culture under a phase contrast microscope. FIGS. 17(a) to 17(d) are microscopic images showing the results obtained by observing the cell encapsulation component 3 days after culture under a phase contrast microscope. FIGS. 18(a) to 18(d) are microscopic images showing the results obtained by observing the cell encapsulation component 7 days after culture under a phase contrast microscope.

Based on the above results, it can be clearly understood that cells encapsulated in the tubular vitrigel could proliferate favorably by culturing.

### Experimental Example 10

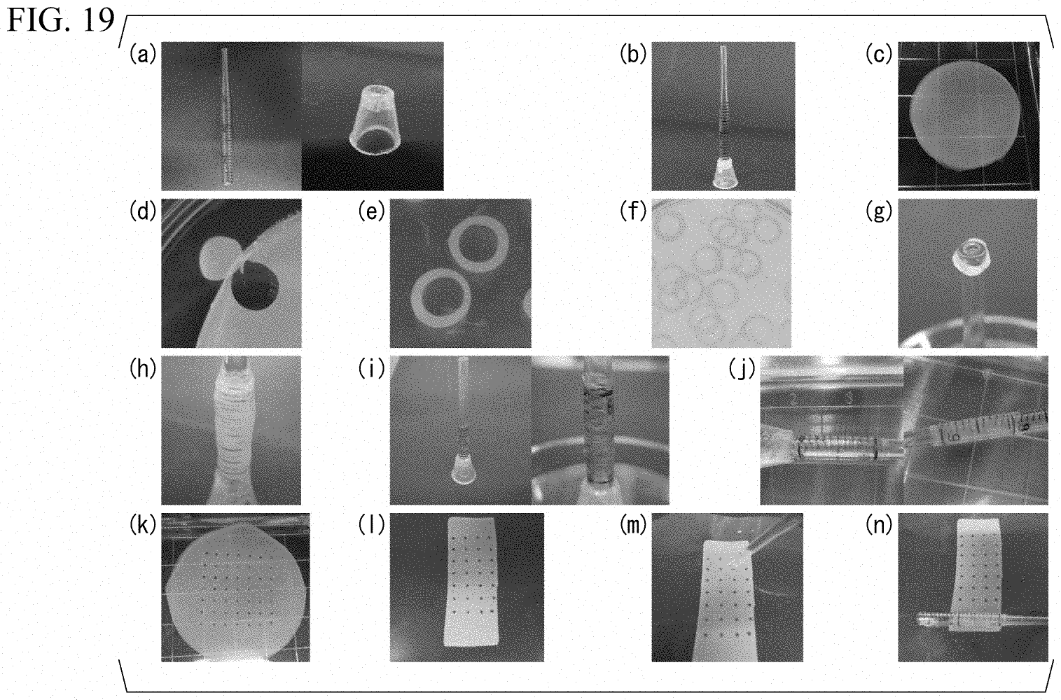

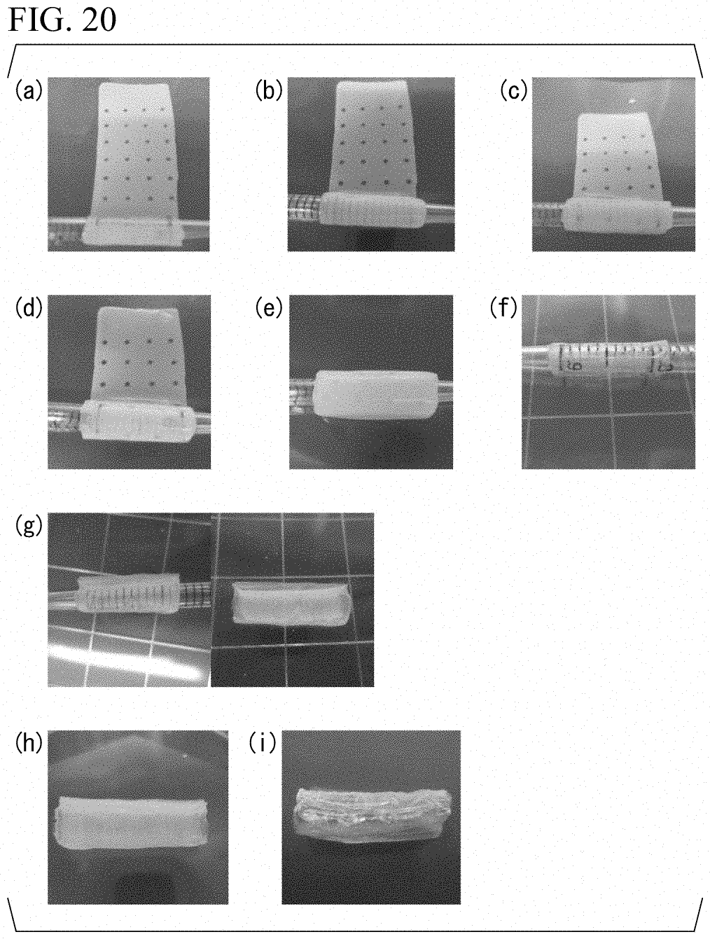

In this Experimental Example, first, a device for producing a tubular vitrigel was produced, and subsequently, a tubular vitrigel in which a sheet-like vitrigel was additionally wound around the outer circumference of the tubular vitrigel was produced. FIGS. 19(a) to 19(n) and FIGS. 20(a) to 20(i) are images showing procedures of this Experimental Example.

### (Production of Device for Producing Tubular Vitrigel)

As shown in FIG. 19(a), a 5 cm tip of a 1 mL pipette (with an outer diameter of 4.6 mm) was cut to form a columnar structure. In addition, 1 cm of a lower end of a 15 mL conical tube was cut, and a hole was formed in the center to form a pedestal that also served as a base. Next, as shown in FIG. 19(b), the columnar structure and the pedestal were sterilized with 70% ethanol, and the both were combined to produce a production device.

### (Production of Tubular Vitrigel)

FIG. 19(c) is an image of the rehydrated atelocollagen vitrigel membrane (collagen amount of 5 mg/cm²). As shown in FIG. 19(d), an atelocollagen vitrigel membrane was cut out using a trepan having a diameter of 5 mm. As a result, a plate-like vitrigel having a diameter of 5 mm was obtained. Next, as shown in FIG. 19(e), the center part of the plate-like vitrigel having a diameter of 5 mm was cut out using a trepan having a diameter of 3.5 mm to obtain a plate-like vitrigel having a through-hole. In the same manner, a total of 32 plate-like vitrigels having through-holes were produced.

Next, 2 mL of a serum-free medium and 2 mL of porcine atelocollagen which were the same as those in Production Example 1 were mixed on ice to prepare a uniform collagen sol. Next, as shown in FIG. 19(f), the above plate-like vitrigel having a through-hole was immersed in the above collagen sol.

Next, as shown in FIG. 19(g), the through-hole of the plate-like vitrigel was penetrated through the columnar structure of the production device. This operation was repeated to obtain a laminate in which a total of 32 plate-like vitrigels were laminated. FIG. 19(h) is an image showing a state in which plate-like vitrigels are laminated.

Next, a small amount of sol around the plate-like vitrigel laminate was removed and gelation was caused in the clean bench at room temperature to obtain a tubular hydrogel. Next, the tubular hydrogel with accompanying the production device was dried and vitrified in the constant temperature and humidity device kept sterile at 10° C. and a relative humidity of 40% to obtain the tubular hydrogel as a dried component as shown in FIG. 19(i). Next, ultraviolet rays were irradiated to the tubular hydrogel dried component. The irradiation amount of ultraviolet rays was 800 mJ/cm² per direction, and irradiation was performed in four directions rotated 90°.

Next, as shown in FIG. 19(j), the tubular hydrogel dried component was rehydrated in PBS and the pedestal was removed.

FIG. 19(k) is an image showing a state in which a sheet-like vitrigel (collagen amount of 5 mg/cm²) having a plurality of through-holes penetrating one surface and the other surface was rehydrated. Next, as shown in FIG. 19(l), the sheet-like vitrigel was cut into a rectangular shape (5 cm×2 cm). Next, as shown in FIG. 19(m), 300 μL of the collagen sol was applied to one surface of the sheet-like vitrigel cut into a rectangular shape.

Next, as shown in FIG. 19(n), the above tubular hydrogel wound around the columnar structure was wound in contact with the surface coated with the collagen sol of the sheet-like vitrigel.

FIGS. 20(a) to 20(e) are images showing procedures of winding a sheet-like vitrigel around the outer circumference of the tubular hydrogel. Next, while facing the end of winding down, the tubular hydrogel with accompanying the columnar structure was dried and vitrified in the constant temperature and humidity device kept sterile at 10° C. and a relative humidity of 40% to obtain the tubular hydrogel as a dried component. FIG. 20(f) is an image showing the tubular hydrogel dried component.

Next, ultraviolet rays were irradiated to the tubular hydrogel dried component. The irradiation amount of ultraviolet rays was 800 mJ/cm² per direction, and irradiation was performed in four directions rotated 90°.

Next, as shown in FIG. 20(g), the tubular hydrogel dried component was rehydrated in PBS and the columnar structure was removed to obtain a tubular vitrigel in which the sheet-like vitrigel was additionally wound around the outer circumference of the tubular vitrigel.

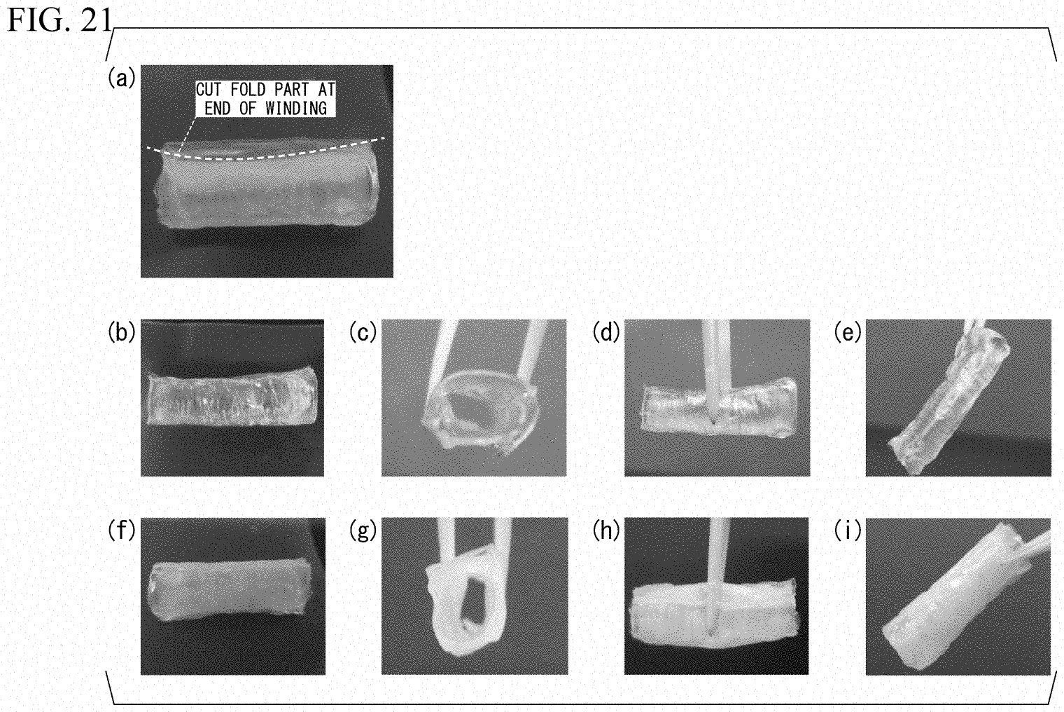

FIG. 20(h) is an image of the obtained tubular vitrigel. FIG. 20(i) is an image of a tubular vitrigel dried component in which the tubular vitrigel was dried and vitrified in the constant temperature and humidity device kept sterile at 10° C. and a relative humidity of 40%. FIGS. 21(a) to 21(i) are images of the produced tubular vitrigel. As shown in FIG. 21(a), rehydration was performed and the fold part at the end of winding of the tubular vitrigel was cut and removed. FIGS. 21(b) to 21(e) are images of the tubular vitrigel dried component obtained by drying and vitrifying the tubular vitrigel from which the fold part has been removed in the constant temperature and humidity device kept sterile at 10° C. and a relative humidity of 40%. In addition, FIGS. 21(f) to 21(i) are images of the tubular vitrigel of the hydrated component.

As a result, it can be clearly understood that the tubular vitrigel of Experimental Example 9 could be easily pinched with tweezers regardless of whether it was in a dried component state or a hydrated component state and had favorable handling properties.

### INDUSTRIAL APPLICABILITY

According to the present invention, it is possible to provide a new technology for producing a tubular vitrigel.

### REFERENCE SIGNS LIST

100, 200 Tubular vitrigel
110a, 110b Plate-like vitrigel
110b′ Sol
210 Sheet-like vitrigel
300a, 300b, 500 Production device
310 Base
320 Columnar structure
330 Pedestal
331 End
400 Cell encapsulation component
410 Cell suspension
411 Cell
420 Air
510 Bottom part
520 Side part
530 Container part
540 Columnar jig
ID Inner diameter
OD Outer diameter

1. A tubular vitrigel composed of a laminate in which a plurality of plate-like vitrigels each having a through-hole are laminated in a thickness direction so that the through-holes are continuous.

2. The tubular vitrigel according to claim 1,
wherein irregularities of the outer surface of the tube are larger than irregularities of the inner surface of the tube.

3. The tubular vitrigel according to claim 1 or 2,
wherein the inner diameter of the tube is 5 μm or more.

4. A tubular vitrigel obtained by additionally winding a sheet-like vitrigel around an outer circumference of the tubular vitrigel according to any one of claims 1 to 3.

5. The tubular vitrigel according to any one of claims 1 to 4, containing native collagen or atelocollagen.

6. The tubular vitrigel according to any one of claims 1 to 5, containing a functional material in at least a part.

7. The tubular vitrigel according to claim 6,
wherein the functional material includes a support, a magnetic substance, a fluorescent material, a contrast agent, an immunosuppressive agent, an anti-inflammatory agent, and a differentiation-inducing factor.

8. A device for producing the tubular vitrigel according to any one of claims 1 to 7, comprising a base and a columnar structure that is able to be fixed to the base.

9. The production device according to claim 8,
wherein the outer diameter of the columnar structure is 5 μm or more.

10. The production device according to claim 8 or 9,
wherein a pedestal having an outer diameter larger than that of the columnar structure is additionally provided at a foundation of the columnar structure.

11. A method of producing a tubular vitrigel, comprising:
a process (a) in which, on the columnar structure of the production device according to any one of claims 8 to 10, (i) after a sol is applied to surfaces of a plurality of plate-like vitrigels each having a through-hole, the vitrigels are laminated through the through-holes, or (ii) after a plurality of plate-like vitrigels each having a through-hole are laminated through the through-holes, a sol is applied to surfaces, to obtain a plate-like vitrigel laminate having a surface coated with the sol;
a process (b) in which the sol is gelled to obtain a tubular hydrogel in which plate-like vitrigels constituting the laminate are connected;
a process (c) in which the tubular hydrogel is dried to obtain the tubular hydrogel as a dried component; and
a process (d) in which the tubular hydrogel dried component is rehydrated and removed from the columnar structure to obtain a tubular vitrigel.

12. The production method according to claim 11, further comprising a process in which ultraviolet rays are irradiated to the tubular hydrogel dried component after the process (c) and before the process (d).

13. The production method according to claim 11 or 12, further comprising
a process in which a sheet-like hydrogel having a surface coated with a sol is wound around the laminate after the process (a) and before the process (b), or
a process in which a sheet-like hydrogel having a surface coated with a sol is wound around the tubular hydrogel, and the sol is gelled to obtain a tubular hydrogel after the process (b) and before the process (c), or
a process in which a sheet-like hydrogel having a surface coated with a sol is wound around the tubular hydrogel dried component, the sol is gelled to obtain a tubular hydrogel, and the tubular hydrogel is dried to obtain the tubular hydrogel as a dried component after the process (c) and before the process (d).

14. An artificial tubular tissue composed of the tubular vitrigel according to any one of claims 1 to 7.

15. A cell encapsulation device composed of the tubular vitrigel according to any one of claims 1 to 7.

16. A cell encapsulation component in which a liquid containing cells is retained in the central part of the cell encapsulation device according to claim 15 in a long axis direction, and air is present at both end parts in the long axis direction.
